(19) United States
(12) Patent Application Publication
(10) Pub. No.: US 2024/0085406 A1
(43) Pub. Date: Mar. 14, 2024

(54) MULTI-CHAMBER DEVICE FOR DETECTING PATHOGENS/MOLECULES AND METHODS OF USING SAME

(71) Applicant: Biological Mimetics, Inc., Frederick, MD (US)

(72) Inventors: Stephen J. DOLLERY, South Riding, VA (US); Ke DU, Riverside, CA (US); Mengdi BAO, Rochester, NY (US); Shuhuan ZHANG, Rochester, NY (US); Rui LIU, Pittsford, NY (US); Taralyn J. WIGGINS, Frederick, MD (US); Gregory J. TOBIN, Frederick, MD (US)

(21) Appl. No.: 18/464,940

(22) Filed: Sep. 11, 2023

Related U.S. Application Data
(60) Provisional application No. 63/406,156, filed on Sep. 13, 2022.

(51) Int. Cl. G01N 33/52; C12N 9/22

(52) U.S. Cl. CPC G01N 33/523; C12N 9/22; C12N 2310/20

(57) ABSTRACT

A method of positively detecting a target pathogen/molecule within a fluid sample and related test devices are described. The device includes a plurality of chambers in adjacent, spaced-apart relationship, and a plurality of isolation valves selectively actuatable to control fluid flow into and out of one or more of the plurality of chambers. The method includes providing the fluid sample into a first chamber of the device; opening a first one of the isolation valves to allow the fluid sample to flow from the first chamber into a second chamber, the second chamber containing an amplification reagent; opening a second one of the isolation valves to allow the fluid sample to flow from the second chamber into a third chamber, the third chamber containing a CRISPR/Cas reagent; illuminating the fluid sample in third chamber with light; and determining whether the fluid sample in the third chamber fluoresces in response to the light.

### RELATED APPLICATION

This application claims the benefit of and priority to U.S. Provisional Patent Application No. 63/406,156 filed Sep. 13, 2022, the disclosure of which is incorporated herein by reference as if set forth in its entirety.

### FIELD

The present inventive concept relates generally to test devices and methods and, more particularly, to test devices and methods for detecting molecules.

### BACKGROUND

The coronavirus disease 2019 (COVID-19) pandemic caused by severe acute respiratory syndrome coronavirus 2 (SARS-CoV-2) has spread across the globe and has become a significant threat to human life. Faster, more accessible detection of infection may effectively reduce the spread of the disease and allow patients to be treated in a timely manner. Unfortunately, conventional test platforms may be either expensive, slow, or not sufficiently sensitive for multiple applications. For example, the reverse transcription real-time polymerase chain reaction (RT-PCR) test for SARS-CoV-2 is highly sensitive, but may take several hours to run and may require personnel with specialized molecular training. The reagents for the PCR test cost approximately $5-$10 per test and the thermocycler approximately $20,000-$150,000, depending upon the model. Rapid antigen tests for SARS-CoV-2 can be performed at home by untrained users within 15-20 minutes, but are relatively insensitive and may cost approximately $12 per test.

### SUMMARY

According to some embodiments of the present inventive concept, a method of positively detecting the presence of a target molecule and/or a pathogen (i.e., a bacterium, virus, or other microorganism that can cause disease) within a fluid sample via a device is provided. The device includes a passageway formed within a translucent polymeric body, the passageway including a plurality of chambers in adjacent, spaced-apart relationship. The device also includes a plurality of isolation valves that are selectively operable to control fluid flow into and out of at least some of the plurality of chambers. The method includes providing the fluid sample into a first chamber of the device; opening a first one of the isolation valves to allow the fluid sample to flow from the first chamber into a second chamber, the second chamber containing an amplification reagent; opening a second one of the isolation valves to allow the fluid sample to flow from the second chamber into a third chamber, the third chamber containing a detection reagent, such as a CRISPR/Cas reagent; illuminating the fluid sample in the third chamber with light at a fluorescence excitation wavelength such as blue or ultraviolet; and determining whether the fluid sample in the third chamber fluoresces in response to the light. However, embodiments of the present inventive concept are not limited to the use of light at blue or ultraviolet wavelengths. There can be readouts that are either visible to the naked eye or accentuated with light at other wavelengths.

In some embodiments, the method further includes heating the fluid sample within the second chamber prior to opening the second one of the isolation valves to allow the fluid sample to flow from the second chamber into the third chamber.

In some embodiments, the fluid sample includes a magnetic bead, and the method further comprises opening a third one of the isolation valves to allow the fluid sample to flow from the third chamber to a fourth chamber, wherein a magnet is positioned within the fourth chamber or within the passageway between the third chamber and the fourth chamber. The fourth chamber may contain a reagent configured to undergo a colorimetric change. In some embodiments, the magnet may be in the third chamber to trap molecules from entering the fourth chamber.

In some embodiments, each isolation valve includes a port extending within the body of the device in a direction transverse to the passageway and a rod movably secured within the port and slidable or turnable (i.e., rotatable) between open and closed positions. In some embodiments a port may have a polygonal cross-sectional configuration (e.g., rectangular, triangular, etc.), and the respective rod has a corresponding polygonal cross-sectional configuration such that the rod is snugly movable within the port.

In some embodiments, one or more of the chambers are funnel- or flute-shaped to facilitate fluid flow into an adjacent, downstream chamber.

In some embodiments, each rod is a carbon fiber rod.

In some embodiments, each rod has visual indicia to correlate to a respective chamber to facilitate proper use of the device by a user.

In some embodiments, the body of the device is formed from a polymeric material, such as polydimethylsiloxane (PDMS).

In some embodiments, the plurality of chambers include three chambers. A first one of the chambers is configured to receive an input sample, a second one of the chambers contains an amplification reagent, and a third one of the chambers contains a CRISPR/Cas reagent. In other embodiments, a fourth chamber may be provided that contains a reagent configured to undergo a colorimetric change. In other embodiments, the plurality of chambers include a chamber configured to receive an input sample, and a detection chamber containing a detection reagent configured to undergo a visually detectable change. In some embodiments, the body is configured to be opened such that the fluid sample can be accessed from one of the plurality of chambers. In some embodiments, the body is configured to be opened such that a detection strip can be dipped in the fluid sample or such that a pipette can obtain the fluid sample.

According to some embodiments of the present inventive concept, a method of positively detecting a presence of a target molecule within a fluid sample includes providing the fluid sample into a first chamber of a handheld device, the device including a plurality of chambers in adjacent, spaced-apart relationship, and a plurality of isolation valves selectively operable to control fluid flow into and out of at least some of the plurality of chambers; opening a first one of the isolation valves to allow the fluid sample to flow from the first chamber into a second chamber, the second chamber containing an amplification reagent or other reagent; and opening a second one of the isolation valves to allow the fluid sample to flow onto a detection strip, or into an analytical device, or into a container for transfer to the analytical device.

In some embodiments, a test kit for analyzing animal and/or human samples is provided and includes a test device, and a device for collecting a sample. The test device includes a fluid passageway with a plurality of pre-filled chambers, and a plurality of manually operable isolation valves (e.g., laterally slidable rods, etc.) to control sample flow between adjacent chambers. The test kit may further include a device configured to illuminate the sample with light at a fluorescence excitation wavelength. The test kit may further include a vial containing a substance configured to disrupt a pathogen/molecule in the sample. The test kit may further include a warming device configured to warm the sample. In some embodiments, the warming device is configured to excite a reaction by externally providing activation energy. In some embodiments, the warming device includes a pouch of chemically activatable material.

Embodiments of the present inventive concept are not restricted to diagnostics, and may be used to test, identify or analyze any material for which a field test or point of care (POC) test is desirable. The examples presented herein utilize SARS-CoV-2 and other targets for the detection of nucleic acids. However, embodiments of the present inventive concept are not restricted to the detection of SARS-CoV-2 or nucleic acids.

Embodiments of the present inventive concept are advantageous over conventional diagnostic/test devices because tests can be performed within a short period time, e.g., thirty (30) minutes, by untrained users. Moreover, the test device can be produced inexpensively. The elegant, affordable, and sensitive molecular diagnostic platform of the present inventive concept is a promising tool for POC diagnostics of emerging diseases in resource-limited settings or home-based diagnostics.

It is noted that aspects of the invention described with respect to one embodiment may be incorporated in a different embodiment although not specifically described relative thereto. That is, all embodiments and/or features of any embodiment can be combined in any way and/or combination. Embodiments of the present inventive concept may also be used for “in-device” sample preparation for a subsequent analytical procedure. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail below.

### BRIEF DESCRIPTION OF THE DRAWINGS

The accompanying drawings, which form a part of the specification, illustrate various embodiments of the present inventive concept. The drawings and description together serve to fully explain embodiments of the present inventive concept.

FIG. 1A is a side view of a pathogen/molecule test device, according to some embodiments of the present inventive concept.

FIG. 1B is a rear view of the pathogen/molecule test device of FIG. 1A taken along line 1B-1B.

FIG. 1C is a cross sectional view of the pathogen/molecule test device of FIG. 1B taken along line 1C-1C.

FIG. 1D illustrates the pathogen/molecule test device of FIG. 1C with the uppermost rod laterally moved to an open position.

FIG. 1E is a cross sectional view of a pathogen/molecule test device with multiple fluid passageways, according to some embodiments of the present inventive concept.

FIG. 1F illustrates an example kit containing a pathogen/molecule test device, a warming device, a light configured to emit light at a fluorescence excitation wavelength, according to some embodiments of the present inventive concept, and a holder configured to hold the device during use.

FIGS. 2A-2E illustrate a series/sequence of steps in the fabrication of the pathogen/molecule test device of FIGS. 1A-1C, according to some embodiments of the present inventive concept.

FIG. 3 is a simplified schematic diagram of a recombinase polymerase (RPA) reaction for isothermal amplification of target molecules using forward and reverse oligonucleotide primers that can be provided in the pathogen/molecule test device of FIGS. 1A-1C.

FIG. 4 is a diagram of CRISPR-Cas12a sensing of a target nucleic acid that can be provided in the pathogen/molecule test device of FIGS. 1A-1C.

FIG. 5 illustrates a sequence (left to right) of the controlled flow of a sample through the pathogen/molecule test device of FIGS. 1A-1C, according to some embodiments of the present inventive concept.

FIG. 6A illustrates eight example evaluation reactions (A-H) of an RPA-CRISPR

assay.

FIG. 6B illustrates endpoint fluorescent images of the reactions (A-H) of FIG. 6A illuminated by a 470 nm light with a target input concentration of 100 fM.

FIG. 6C illustrates normalized fluorescence signals of the endpoint reactions of FIG. 6A.

FIG. 7A illustrates endpoint fluorescent images of reactions with a SARS-CoV-2 target and without a target (NC) illuminated by a 470 nm light.

FIG. 7B illustrates a graph of normalized fluorescence signals (normalized intensity versus viral concentration (fM) of the reaction products with and without a SARS-CoV-2 target.

FIG. 8A illustrates endpoint fluorescence images of a reaction with 100 fM of SARS-CoV-2, Influenza, Human RPP30 gene, MERS-CoV, and SARS-CoV targets.

FIG. 8B is a graph that illustrates normalized fluorescence signals of the reactions with 100 fM of SARS-CoV-2 (purple), Influenza (blue), human RPP30 gene (green), MERS-CoV (orange) and SARS-CoV (red).

FIG. 9A is a graph that illustrates normalized intensity of a CRISPR reaction containing 1 nM SARS-CoV-2 incubated inside a microtube versus the pathogen/molecule test device of FIGS. 1A-1C.

FIG. 9B is a graph that illustrates a normalized intensity of an RPA-CRISPR reaction containing 100 fM SARS-CoV-2 incubated inside a microtube versus the pathogen/molecule test device of FIGS. 1A-1C.

FIG. 10A is a graph that illustrates a normalized intensity of a CRISPR reaction containing 1 nM SARS-CoV-2 incubated by a hot plate versus a hand warmer.

FIG. 10B is a graph that illustrates a normalized intensity of an RPA-CRISPR reaction incubated by a hot plate versus a hand warmer.

FIG. 11 is a schematic illustration of a pathogen/molecule test device, according to some embodiments of the present inventive concept.

### DETAILED DESCRIPTION

The term “FAST”, as used herein, is an abbreviation for a “funnel-adapted sensing tube”, multi-chamber reaction device according to embodiments of the present inventive concept. The term “PDMS”, as used herein, is an abbreviation for polydimethylsiloxane, which is a plastic polymer from which a device according to the present inventive concept can be made. The term “RPA”, as used herein, is an abbreviation for recombinase polymerase, an isothermal process to amplify target molecules to increase sensitivity. The term “PCR”, as used herein, is an abbreviation for polymerase chain reaction, a thermal cycling process to amplify target molecules to increase sensitivity. The term “dNTP”, as used herein, is an abbreviation for deoxynucleoside triphosphate (dATP, dCTP, dGTP, and dTTP), the building blocks of DNA used in polymerase amplification reactions. The term “CRISPR”, as used herein, is an abbreviation for clustered regularly interspaced short palindromic repeats, which are the hallmark of a bacterial defense system that forms the basis for CRISPR-Cas9 genome editing technology.

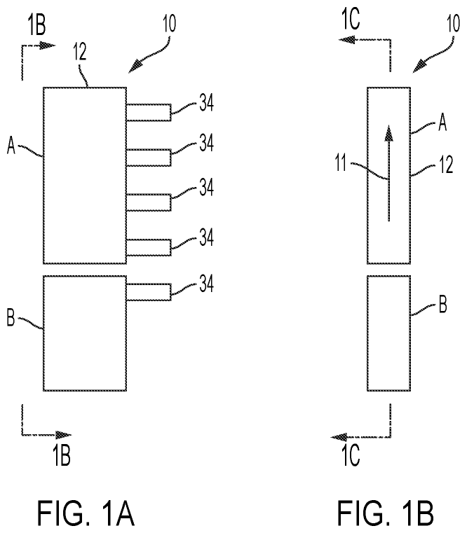

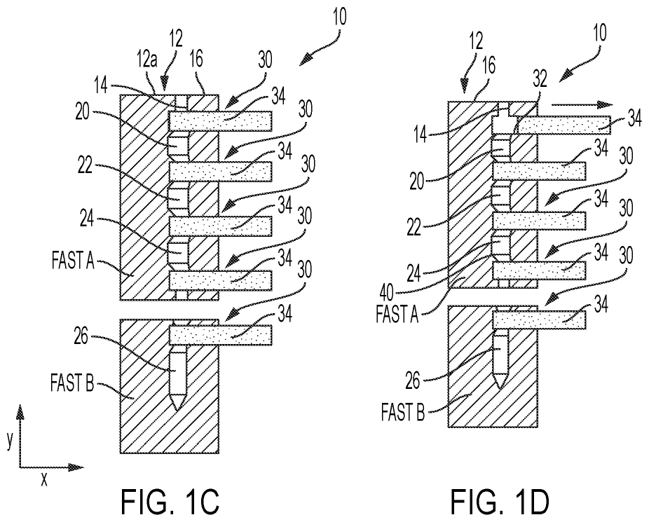

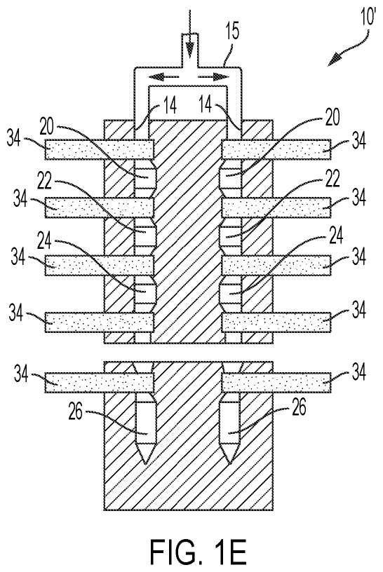

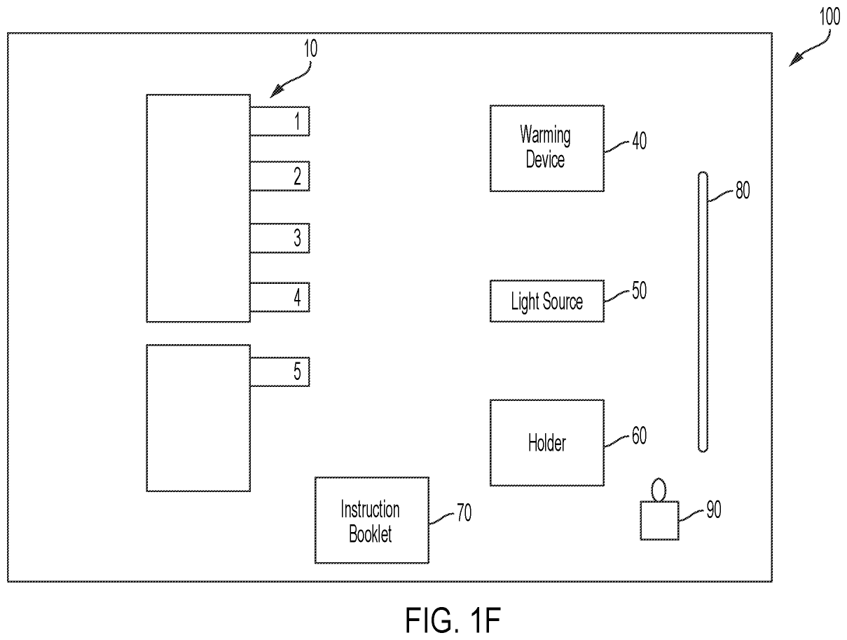

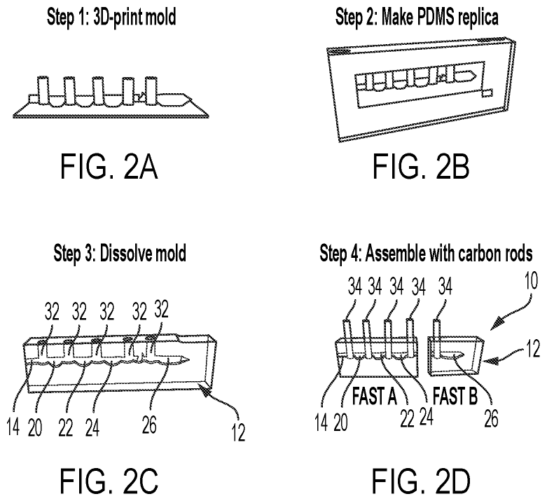

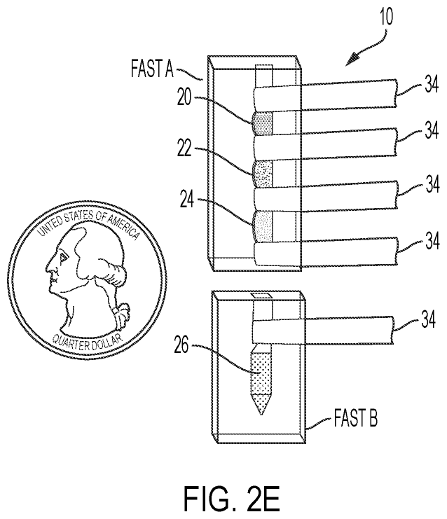

Referring initially to FIGS. 1A-1C and FIGS. 2C-2D, a handheld (i.e., small, compact, can be held in a person's hand) pathogen/molecule test device 10, according to some embodiments of the present inventive concept, is illustrated. The device 10 includes an elongate body 12 with an elongate fluid passageway 14 extending within the body 12. The body 12 may be formed from a translucent or transparent polymeric material, such as PDMS. The translucent or transparent polymeric material facilitates viewing of the interior of each chamber 20, 22, 24, 26. In some embodiments, the polymeric material is a semi-soft material which prevents or reduces leaks at the isolation valves 30 described below. However, various other translucent or transparent materials may be utilized.

The passageway 14 includes an inlet 16 in a first (e.g., upper) portion 12a of the body 12, and a plurality of chambers 20, 22, 24, 26 in adjacent, spaced-apart relationship. The passageway 14 is configured for controlled, sequential passage of a fluid sample therethrough and into the chambers 20, 22, 24, 26, as will be described below. A plurality of isolation valves 30 are operably associated with the passageway 14 and are individually actuatable to control fluid flow into and out of the chambers 20, 22, 24, and into chamber 26.

In the illustrated configuration, each chamber 20, 22, 24 has a generally funnel or flute shape (i.e., each chamber has a downwardly converging configuration) to facilitate fluid flow into an adjacent, downstream chamber. The final chamber 26 is longer than the other chambers 20, 22, 24, as illustrated. However, the chambers 20, 22, 24, 26 may have various shapes and configurations and are not limited to the illustrated shape/configuration. Each chamber 20, 22, 24, 26 can have a different shape, also.

Each isolation valve 30 includes a port 32 extending within the body 12 in a direction transverse to the passageway 14 and in communication with the passageway 14, as illustrated in FIG. 1C. A rod 34 (FIGS. 1A, 1C, 2D) is movably (e.g., slidably) secured within each respective port 32 and is laterally movable between open and closed positions. The rods 34 can be moved within a respective port 32 to control and block flow from one chamber to another. When in a closed position, each rod 34 extends across the passageway 14 and blocks the flow of fluid. When a rod is slightly or entirely withdrawn from the body 12 (i.e., moved to an open position), fluid can flow past the rod 34 within the passageway 14. FIG. 1D illustrates the uppermost rod 34 laterally moved to an open position such that a fluid sample can flow down the passageway 14 into the first chamber 20. The remaining rods 34 in FIG. 1D are in a closed position. Each rod 34 may be formed from carbon fiber; however, other materials may be used, such a polymeric materials and metallic materials. Each rod 34 may have visual indicia (a color, a pattern, a character, etc.) to correlate to a respective chamber to facilitate proper sequential use of the device by a user. For example, all or a portion of each rod 34 may have a different respective color or a different respective visual pattern than the other rods 34. A sequence of actuation of the valves 30 may be “open and close the red rod 34 first, then open and close the green rod 34, then open and close the blue rod 34. The rods 34 can be numbered (e.g., sequential numbering 1, 2, 3, etc., as illustrated in FIG. 1F) in addition or in lieu of color coding.

Embodiments of the present inventive concept are not limited to the illustrated configuration and operation of the rods 34. The rods 34 may have various configurations and modes of operation. For example, in some embodiments, a rod 34 may contain an aperture therethrough such that, when the rod 34 is in a closed position, the rod is positioned such that the aperture is not in fluid communication with the passageway 14. As such, flow of a fluid sample down the passageway 14 is blocked. When the rod 34 is moved to an open position, the rod is positioned such that the aperture is in fluid communication with the passageway 14 and such that a fluid sample can flow down the passageway 14 through the aperture.

In addition, numerous alternative valve systems can be used in place of rods to achieve opening and closing of the channels including valves that can be actuated by pushing, pulling and rotating. Embodiments of the present inventive concept are not limited to the illustrated isolation valves.

The illustrated device 10 includes a single passageway 14 with a plurality of chambers 20, 22, 24, 26. As will be described below, the first chamber 20 is configured to receive an input sample, the second chamber 22 contains an amplification reagent, the third chamber 24 contains a CRISPR/Cas reagent, and the fourth chamber 26 may include a reagent configured to undergo a colorimetric change. Thus, the illustrated device 10 is integrated with RPA amplification and CRISPR cleavage for power-free, pipette-free, and sensitive nucleic acid detection.

In other embodiments, the body 12 of the device 10 may contain two or more passageways 14 to enable the testing of multiple samples in the same device 10, as illustrated in FIG. 1E. In FIG. 1E, device 10′ includes two respective passageways 14, each in fluid communication with a manifold 15, as illustrated. The illustrated device 10′ is designed such that 50% of a sample flows into each of the respective passageways 14. Such an expanded device 10′ allows a single sample to be tested for two pathogen/molecule targets. Other devices with more fluid passageways, such as three, four, five or more will allow testing for multiple pathogen/molecule targets. In addition, the number of chambers along any respective fluid passageway 14 may vary. For example, a device with less than 4 chambers or more than 4 chambers for a respective fluid passageway 14 can be utilized, depending on the application.

Referring to FIGS. 2A-2E, an example fabrication process for the device 10 that is rapid, inexpensive, and environmentally friendly will be described. Initially, as shown in FIG. 2A, a mold is printed by a 3D printer, such as a Flashforge® 3D printer, using a water-soluble polyvinyl alcohol (PVA) resin. The mold represents the hollow-spaces of the body 12 which include the passageway 14, the chambers 20, 22, 24, 26, and the valve ports 32. The 3D shape of the PVA mold can be designed by a computer aided design program, such as AutoCad®. 3D printing is advantageous because it allows the fabrication of a PVA mold with 200 μm resolution, and allows the configuration and interior dimensions of the passageway 14 and the chambers 20, 22, 24, 26 to be custom-designed.

In some embodiments, one or more of the chambers 20, 22, 24, 26 are sized to hold between 50 and 100 microliters (approximately 1 to 3 drops) of liquid. However, other sizes may be utilized. In the event that smaller or larger chambers are desired, the program for printing the 3D mold can be adjusted accordingly. An exemplary 3D printer that can be used to make the mold has an adjustable printing speed between 10 to 100 mm/s with a nozzle diameter of 0.4 mm, and a maximum operation temperature of 240° C. Each layer may have a thickness less than 0.4 mm, although other thicknesses may be utilized.

The mold is then placed into a container (e.g., an aluminum container) for casting the body 12 using PDMS, or a similar composition that permits visualization of a positive detection, as illustrated in FIG. 2B. A silicone elastomeric base (e.g., PDMS) and curing agent (SYLGARD® 184 Silicone Elastomer Kit) with a ratio of 10:1 may be mixed and poured into the injection mold. Once filled with PDMS, the container is placed into a desiccator for approximately 2 hours to remove any air trapped inside and then baked in an oven at 65° C. for approximately 24 hours. After curing, the mold is taken out from the container and the PVA core is dissolved (FIG. 2C) with deionized water under sonication, leaving only the body 12 behind. In one embodiment, the body 12 is cut into two individual pieces, FAST A and FAST B, to prevent backflow of the Cas complex into the FAST A device which may unintendedly cleave the reporter molecules. It should be noted that, although the device 10 can be made using a mold that is dissolved, embodiments of the present inventive concept are not limited thereto. In other embodiments, the device 10 could be made by injection molding or 3D printing.

Rods 34, such as carbon fiber rods, are inserted into the ports 32, separating and residing between the chambers 20, 22, 24, 26. The rods 34 are designed to fit tightly within the ports 32. In some embodiments, the outer diameter of the rods 34 is 3.5 mm. However, rods 34 having different diameters may be utilized in other embodiments. The assembled device 10 is illustrated in FIG. 2D. The assembled device 10 can be sterilized by a UV Sterilizer Cabinet and stored under room temperature for later use. The transparent nature of the PDMS facilitates viewing of the interior of each chamber 20, 22, 24, 26.

The assembled device 10 can be pre-loaded with reagents for the various reactions. FIG. 2E illustrates the assembled device 10 with colored water within the chambers 20, 22, 24, 26 representing the contents of the chambers 20, 22, 24, 26, for ease of illustration. Chamber 20 is used to hold the input sample to be tested. Chamber 22 contains an amplification reaction reagent, and chamber 24 contains a detection reaction reagent.

In some embodiments, chamber 22 can be pre-loaded with a solution containing deoxynucleotide oligo primers which are specific to the target sequences and other reagents and buffers for the RPA amplification step. The volume of the RPA reaction mixture can be in a range of 20-150 μL, such as 20, 30, 40, 50, 60, 70, 80, 90, 100, 110, 120, 130, 140, or 150 microliters or other convenient volume with a preferred volume of 30 to 100 microliters. Reagents such as those from TwistDx Ltd. (Cambridge, UK) can be used for the RPA reaction. In one embodiment, chamber 24 can be pre-loaded with reagents for the CRISPR-Cas detection step. In a further embodiment, Cas12a, available from Integrated DNA Technologies (Coralville, IA), can be utilized. The crRNA, which functions as the guide sequence to sense the target nucleic acid, can be designed using methods known in the art and obtained from a vendor that provides synthetic RNA, such as Integrated DNA Technologies.

Operation of the pathogen/molecule test device 10 is relatively simple and requires no specialized equipment or training. The process can be broken into the following series of steps that can be used by individuals with little training in medical technology:

Step 1. Open kit and read instructions. The kit contains a sealed sterile cotton-tipped applicator for collecting the sample, a small flexible plastic dropper vial containing a substance for disrupting the pathogen/molecule in the sample, the device 10, an optional warming device that operates by exothermal chemical reaction (e.g., a hand warmer), an optional light source for visualizing the detection results, and instructions for use. FIG. 1F illustrates an example kit 100 containing a pathogen/molecule test device 10, a warming device 40, such as a hand warmer/foot warmer, a light source 50 configured to emit light at a fluorescence excitation wavelength, an instruction booklet 70, a sealed sterile applicator 80 for collecting the sample, and a dropper vial 90 containing a substance for disrupting the pathogen/molecule in the sample. In addition, the kit 100 may include a holder 60 configured to hold the device 10 in the proper orientation during use.

Step 2. Activate the hand warmer, if the diagnostic is used in cold ambient temperatures.

Step 3. Collect the sample. Open the envelope containing the cotton-tipped applicator and collect the sample. For respiratory pathogens, swirl the applicator inside each nostril for approximately 15 seconds. For oral pathogens, collect the sample by wiping the applicator upon the inner surfaces of the mouth including the tongue. For urinary tract pathogens, collect a small volume of urine in a cup or glass. Insert the applicator into the urine to collect the sample.

Step 4. Release the nucleic acid. Place the cotton-tipped applicator into the small dropper vial. Rotate the applicator while squeezing and releasing the vial several times. Remove and discard the applicator. Depending on the pathogen/molecule to be detected, shake the vial to mix well.

Step 5. Introduce the sample. Unwrap the device 10 and hold it such that the long dimension is vertical, indicated by indicia on the body 12, such as the arrow 11 in FIG. 1B. Slide the top rod 34 relative to the body 12 to an open position. Express one or more drops of disrupted sample into the top chamber 20 of the device 10. Slide the top rod 34 back into the body 12 to the closed position.

Step 6. Initiate the target amplification reaction. While holding the device 10 vertical, slide the second rod 34 to an open position to allow the sample to flow into the second chamber 22 containing the amplification reagents. Move all rods 34 back to respective closed positions and place the device 10 on the hand warmer 40 for 10 minutes for target amplification. A low-cost hand warmer disposable pouch (self-contained exothermal reaction) may be used as a heating source for the entire reaction. The term “hand warmer” is intended to include a “foot warmer” and includes any type pouch of chemically activatable material.

Step 7. Initiate the CRISPR/Cas detection reaction. While holding the device 10 vertical, slide the third rod 34 to an open position to allow the sample to flow into the third chamber 24 containing the CRISPR/Cas reagents. A holder may be provided to hold the device 10 or the device 10 can be held directly by a user. Move all rods 34 back to respective closed positions and place the device 10 on the hand warmer for 10 minutes for the detection reaction.

Step 8. Visualize the results. Shine blue-light at the device 10 via the light 50. A positive result is indicated by a glowing liquid. With blue light illumination, the fluorescence signal can be detected by the naked eye in the dark.

A variety of light-emitting or colorimetric readouts can be used to signal a positive detection of the target molecules. In some embodiments, the single stranded DNA molecule (ssDNA) to be cleaved by the activated CRISPR/Cas molecule is covalently linked to a fluorophore on one end and a quenching molecule on the other. When the ssDNA is cleaved, the fluorophore is released and a fluorescent light is visible under illumination by blue light. In an additional embodiment, the ssDNA can be linked to an enzyme such as alkaline phosphate or hydrogen peroxidase on one end and a magnetic bead on the other. In embodiments that include the magnetic bead, a magnet 40 is included in the third chamber 24 or directly upstream of the fourth chamber 26, for example, in the passageway between the third chamber 24 and the fourth chamber 26. After the CRISPR/Cas reaction, the solution is passed by a magnet to capture ssDNAs linked to the magnetic bead. If the reaction is negative, meaning that no target pathogen/molecule was detected, the enzyme remains attached to the ssDNA and is bound to the magnet. If the reaction is positive, meaning that the target pathogen/molecule was detected, the enzyme is cleaved from the ssDNA with the magnetic bead and can flow into a chamber (e.g., chamber 26) for detection. In some embodiments, detection is by a fluorophore, in others by a colorimetric chemical, or other detection method that allows detection of a positive signal by light or by a change in color upon reaction with the released enzyme.

In some embodiments, the body 12 of the device 10 is configured to be opened such that the fluid sample in the one of the chambers in the body 12 (e.g., FIG. 1C) can be accessed so that a detection strip can be dipped in the fluid sample or such that a pipette can obtain the fluid sample for transfer to an analytical device. In some embodiments, one of the isolation valves can be opened such that the fluid sample can flow onto a detection strip, or into an analytical device, or into a container for transfer to the analytical device. For example, the fourth valve in the embodiment of FIG. 1C could be opened such that the fluid sample in the third chamber 24 is allowed to flow onto a detection strip, or into an analytical device, or into a container for transfer to the analytical device.

Cas12a nuclease cleaves ssDNA molecules. In some embodiments, virus RNA genomes are copied into ssDNA targets using reverse transcriptase. To make copy DNA (cDNA), the virus is disrupted to release the RNA and mixed in a buffered solution which includes any one of many reverse transcriptases, random hexamer oligonucleotide primers, and dNTPs. The reaction is incubated from 10 to 60 minutes between 25° C. and 37° C. The reverse transcription reaction can take place in the first chamber, if needed. In other embodiments, the DNA genome of the pathogen is detected without reverse transcription.

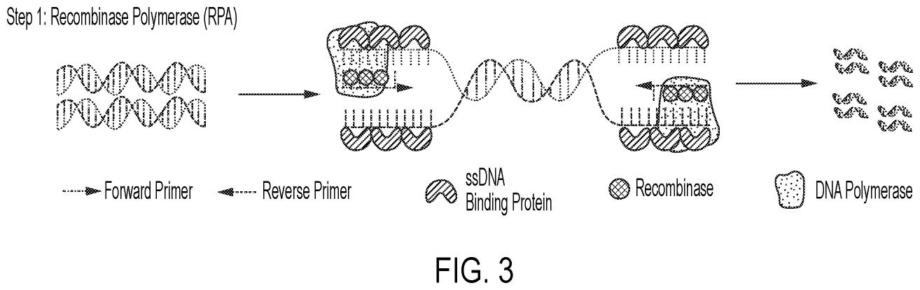

To increase the sensitivity of detecting target molecules that are at low concentrations, any number of methods to amplify the target molecules known in the art can be employed. The isothermal amplification step described above is used to geometrically increase the number of target molecules without the need for thermal cycling. FIG. 3 illustrates the amplification step in which the target molecule on the left is mixed with forward and reverse oligonucleotide primers and the amplification complex consisting of deoxynucleic triphosphates (dNTPs), ssDNA binding protein, a recombinase, and a polymerase. After a short incubation between room temperature and 37° C., multiple copies of the original target molecules have been synthesized.

PCR is an alternative method of amplification. For PCR, the solution containing the target molecules is mixed with the forward and reverse primers, dNTPs, a temperature-stable polymerase such as Taq, and buffer components. The reaction undergoes multiple cycles such as 94° C. denaturation for 1 minute, 55° C. primer annealing for 1 minute, and 72° C. polymerase extension for 0.5 minute. The number of cycles selected depend upon the amount of amplification desired and is typically between 10 and 30 cycles.

The CRISPR/Cas system evolved in bacterial cells to protect themselves from infection by bacteriophages. When a colony of bacteria is infected, survivors may produce crRNAs which are complementary to DNA or RNA sequences that are unique to the bacteriophages. These crRNAs combine with Cas proteins to form an effector complex. If a bacteriophage bearing the same sequence as the initial infecting agent enters the cell and uncoats and the crRNA binds it cognate complementary target sequence, the Cas molecule is activated to indiscriminately digest nucleic acids, thereby cleaving the bacteriophage genomic material. A variety of Cas molecules have been discovered that sense and cleave single-stranded RNA or DNA or double-stranded DNA. As an example, Cas12a senses dsDNA and cleaves single stranded DNA. Embodiments of the present inventive concept are not limited to the use of Cas12a. In contrast, Cas13a could be used for detecting single stranded RNA molecules such as unamplified viral genomes.

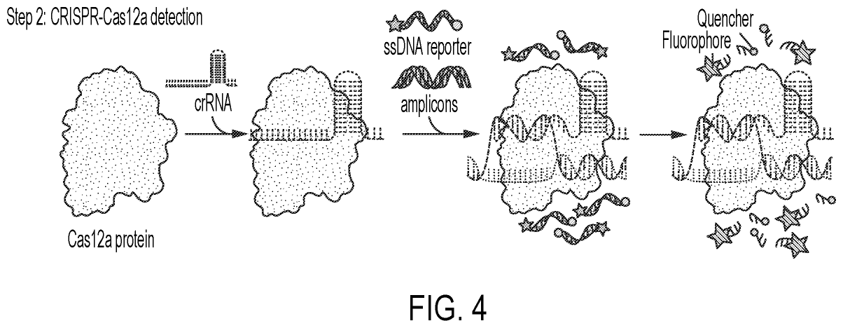

FIG. 4 illustrates CRISPR-Cas12a detection. In the first step, a synthetic crRNA molecule which contains sequences complementary to the target sequence is mixed with the Cas molecule to form a complex. When the complex encounters amplified target molecules, the nuclease activity of the Cas is activated and it cleaves single-stranded DNA molecules. In this example, the single-stranded DNA molecules have covalently linked fluorescent reporter molecules on one end and a quenching molecule on the other. Cleavage of the DNA tether releases the reporter from the quench to enable visible detection of the reporter. Similarly, Cas13a can be used to sense or detect single-stranded RNA molecules such as those released by coronaviruses, influenza viruses, picornaviruses, and many others without conversion to DNA in an amplification process.

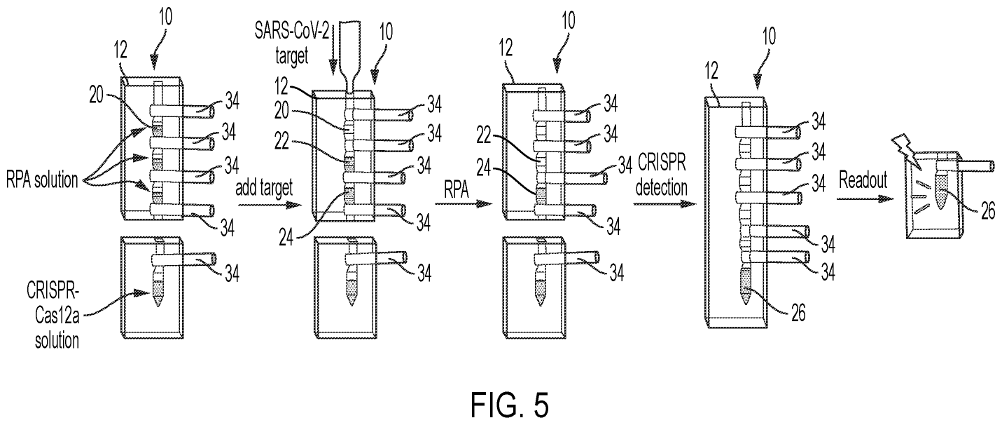

FIG. 5 illustrates the flow of a sample through the device of FIGS. 1A-1C. One or more drops of sample is introduced into an open chamber 20 at the top of the device 10. The first rod 34 is then pushed back in to the body 12 to prevent backflow of the sample from the device 10. The rod 34 beneath the chamber 20 is then slidably moved to an open position to allow the sample to flow into the next chamber 22, which contains the RPA amplification reaction mixture. After shaking the device 10 to mix the sample, an incubation period of approximately 5 to 20 minutes allows target amplification. The next rod 34 is slidably moved to an open position to allow the sample to flow sequentially into the next chamber 24 containing the CRISPR/Cas detection system with the ssDNA reporter molecules. If the Cas detects the target, the reporter and quench molecules are unlinked and a light-emitting readout is visible. In the example shown in FIG. 5, a fluorescent reporter molecule is used which can be seen by the naked eye by shining blue light at the device. Alternative methods of detection can also be used, however.

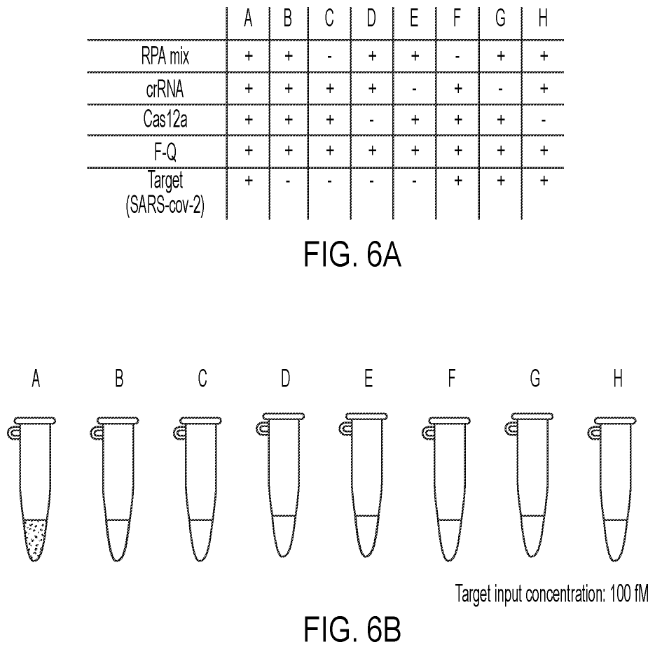

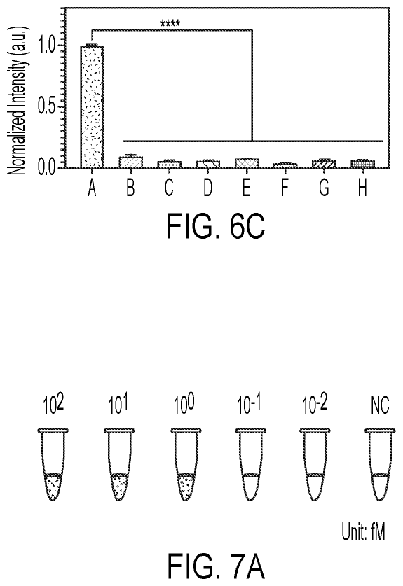

FIGS. 6A-6C illustrate evaluation reactions of an RPA-CRISPR assay. In FIG. 6A, eight sample tubes (labeled A through H) contain the various illustrated combinations of reagents and are designed to assess signal production when one or two components are omitted. FIG. 6B illustrates light emitted from the eight sample tubes. When all components are present, as in Sample A (FIG. 6B), light is emitted. No signal is observed when any one or two components are omitted, as illustrated in Samples B-H of FIG. 6B. FIG. 6C represents the numerical light emission when the samples are analyzed in a fluorometer.

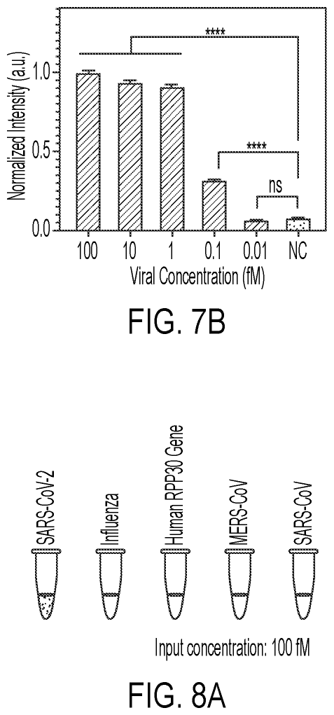

FIGS. 7A-7B illustrate an evaluation of sensitivity of detection. The RPA-CRISPR assay was assembled to detect a fragment of the SARS-CoV-2 Spike gene (SEQ 001). To produce a target RNA, a DNA fragment of the Spike gene was synthesized and cloned into a pUC57 vector. Run-off RNA transcripts of approximately 800 bases in length were synthesized using T7 RNA polymerase to be of the same sense as coronavirus genomic RNA. After purification, the concentration of the RNA was determined using spectroscopy at 260 nm. A series of 10-fold dilutions were produced to assess the sensitivity of the detection system. The first tube contained 100 μl of a solution of 10² femtomolar (fM) concentration. The other tubes contained 10-fold less concentrated target RNA. cDNA was synthesized from the target RNA samples and the products of these reactions were subjected to RPA. After amplification for 10 minutes and CRISPR/Cas reaction for 10 minutes, the signal was detected visually using a blue-light flashlight emitting at approximately 470 nm. During the cDNA, RPA amplification, and CRISPR/Cas reactions, the device 10 was placed on a hot plate and warmed to 37° C. FIG. 7A shows a strong signal at 1 fM and a weak signal at 0.1 fM concentrations. FIG. 7B shows a graph of the samples analyzed in a fluorometer and confirms that the faint signal from the 0.1 fM sample is statistically greater than the negative control.

Several measures can be adopted to increase the sensitivity of the diagnostic. For example, multiple crRNA sequences can be combined in the CRISPR complexes, each representing a different locus of the genomic nucleic acid. Upon release of the nucleic acid, naturally-occurring or introduced nucleases digest the RNA or DNA into fragments, each of which can result in an independent signal. Thus, if four crRNA sequences are used in combination and each sense a different portion of the same RNA target (or amplified DNA target), the signal is effectively amplified 4-fold.

The specificity of the diagnostic assay is largely a function of the design of the crRNA molecules to ensure that they bind to sequences that are unique to the intended target molecules. In the case where all types of influenza are to be detected, for example, the crRNA guide sequences would be derived from viral sequences common to all types of influenza. In the case where a specific type of influenza is to be detected, such as H1N1, crRNA sequence would be derived to be specific to the sequences of the intended target(s). For coronaviruses, the test can be designed to diagnose all human coronaviruses, including SARS-1 and SARS-CoV-2; only SARS-CoV-2; or, more specifically, a single strain of SARS-CoV-2. Thus, by designing the crRNA sequence to be specific to a target sequence of a single strain or isolate, a series of diagnostics can be designed to type the infectious material without the need for sequence analysis.

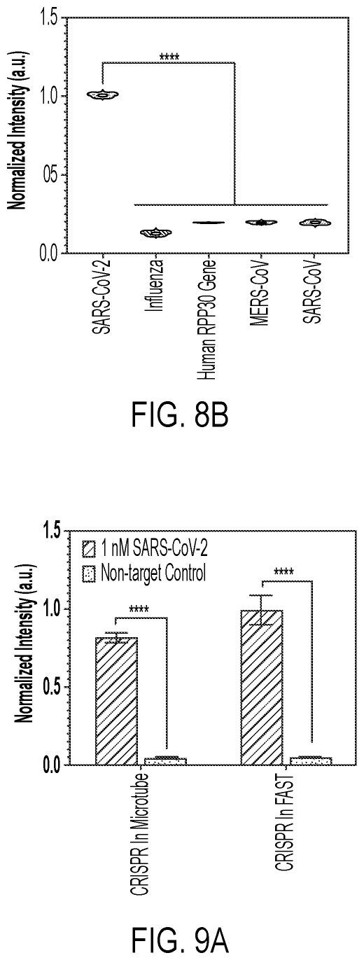

FIGS. 8A-8B show a test of the specificity of detection of the SARS-CoV-2 diagnostic. dsDNA sequences specific to SARS-CoV-2, influenza (which), the human RPP30 gene, MERS-CoV, or SARS-CoV-1 were assayed at a concentration of 100 fM target molecule. The molecules were amplified for 10 min by RPA and mixed with the CRISPR-Cas12a-SARS-CoV-2 complex. FIG. 8A shows light output when illuminated using a blue-light flashlight. FIG. 8B shows the fluorometry-derived data. FIGS. 8A and 8B clearly show that the intensity of the signal from the SARS-CoV-2 target molecules is far greater than the signals from the non-specific targets.

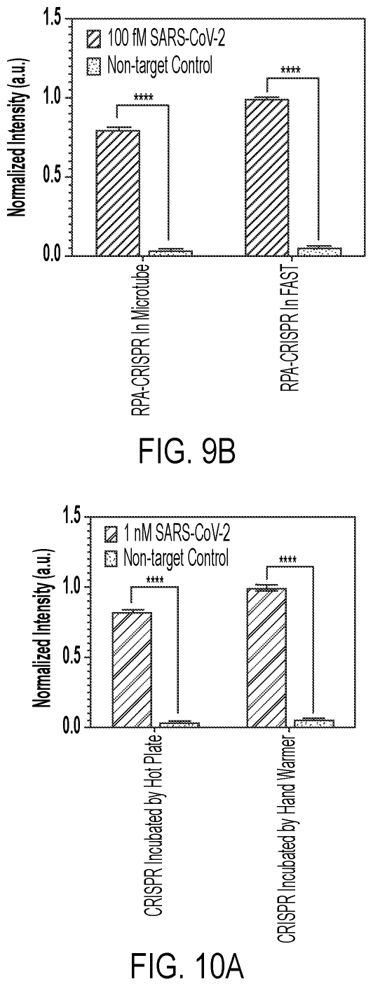

FIG. 9A illustrates a CRISPR reaction incubated inside microtubes versus the device 10 of FIGS. 1A-1C. FIG. 9B illustrates a RPA-CRISPR reaction incubated inside microtubes versus the device 10 of FIGS. 1A-1C. In FIG. 9A, the RPA step was performed in tubes and then placed into either tubes or the device 10 of FIGS. 1A-1C for the CRISPR-Cas step. Light emission was quantitated by fluorometry at 470 nm. The samples containing 1 nM SARS-CoV-2 target RNA both demonstrate greater signals than when a non-target control is used with a p value <0.0001 from an unpaired t-test. Similarly, FIG. 9B shows the results when both the PRA and CRISPR reactions are performed either in tubes or in the device 10 of FIGS. 1A-1C. Although the improvement in signal is not statistically significant in the two samples performed in the device 10 compared to tubes, the trend is that the device 10 produces slightly higher signals. Thus, the reactions described above can be performed in the device 10 without loss of signal.

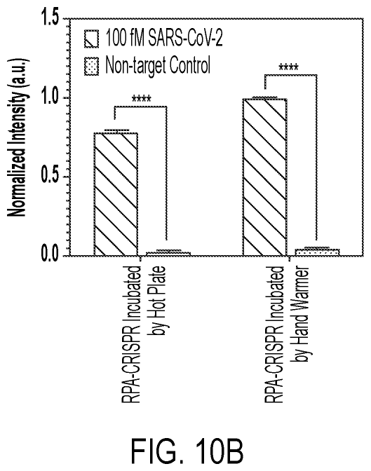

Both the amplification and the CRISPR/Cas reactions proceed more rapidly at 37° C. than at room temperature. FIGS. 10A-10B compare the light emission of samples where the CRISPR or both the amplification and CRISPR steps were performed on either a hot plate of warmed with a simple hand warmer. FIG. 10A shows the differences in light output signal when the CRISPR step is incubated on either a hot plate or by a hand warmer and FIG. 10B shows the signal when both steps are incubated on a hot plate or hand warmer. In both cases the use of the hand warmer provides a small, but detectable increase in signal, likely due to better thermo-conductivity.

In the embodiments described above, a fluorophore is covalently linked to a quenching molecule by a single stranded DNA molecule. When the CRISPR/Cas molecule is activated by sensing the intended target molecule, its nuclease activity cleaves the DNA to release the fluorophore from the quench to develop the signal of a positive reaction. Numerous alternatives for the signal can be used and are known to the art. For example, a colorimetric reaction can be substituted for the fluorophore-quench methodology. In one embodiment, the sample passes through the amplification and CRISPR/Cas chambers 22, 24 of the device 10, as described above. However, instead of the DNA to be cleaved being cross-linked to fluorophore and quench molecules, it can be linked to an enzyme that produces a colorimetric reaction and a magnetic bead. Such an enzyme can be horseradish peroxidase, alkaline phosphatase, or other enzyme known to the art. After the CRISPR/Cas reaction has been incubated for the specified period of time, the device 10 is held vertically and the fourth rod 34 is slidably moved to an open position to allow the fluid to move into the fourth chamber 26 containing a magnet. The magnet will bind to all uncleaved detector molecules. After a brief shake and incubation, the fifth rod 34 is slidably moved to an open position to allow detector molecules that have been cleaved from the magnetic beads to flow into a final chamber containing a colorimetric reagent. Alternatively, the magnet can be placed within or adjacent to the orifice between chamber 24 and 26. The reagent is selected based upon the selection of the color-inducing enzyme. For horseradish peroxidase it could be TMB (3,3′,5,5′-tetramethylbenzidine) which yields a blue color upon reaction with HPR. For alkaline phosphatase is could be PNPP (p-nitrophenyl phosphate) which produces a yellow color upon reaction with AP.

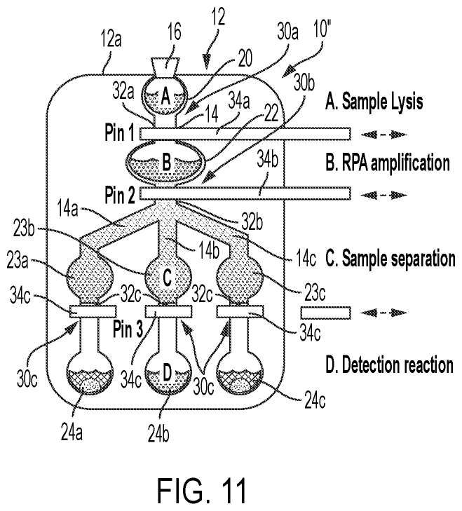

FIG. 11A is a schematic illustration of a handheld pathogen/molecule test device 10″, according to some embodiments of the present inventive concept. The device 10″ includes a body 12 with a fluid passageway 14 that branches into three separate passageways 14a, 14b, 14c. The passageway 14 includes an inlet 16 in a first (e.g., upper) portion 12a of the body 12 and first and second downstream chambers 20, 22 in adjacent, spaced-apart relationship. Passageways 14a, 14b, 14c each include respective third and fourth chambers 23a, 24a, 23b, 24b, 23c, 24c in adjacent, spaced-apart relationship, as illustrated.

A plurality of isolation valves 30a, 30b, 30c are operably associated with the passageways 14, 14a, 14b, 14c and are individually actuatable to control fluid flow into and out of the chambers 22, 23a, 23b, 23c, 24a, 24b, 24c. Each isolation valve 30a, 30b, 30c includes a respective port 32a, 32b, 32c extending within the body 12 in a direction transverse to the passageways 14, 14a, 14b, 14c and in communication with the passageways 14, 14a, 14b, 14c. A respective rod 34a, 34b, 34c is movably (e.g., slidably) secured within each respective port 32a, 32b, 32c and is laterally movable between open and closed positions. The rods 34a, 34b, 34c can be moved within a respective port 32a, 32b, 32c to control and block fluid flow, as described above with respect to FIGS. 1A-1E.

The first chamber 20 in passageway 14 is configured to receive an input sample, and the second chamber 22 in passageway 14 contains an amplification reagent. Amplification is performed in the single chamber 22 with multiplexing oligo primers. The amplified material is then separated into the three passageways 14a, 14b, 14c and respective third chambers 23a, 23b, 23c below. The fourth chambers 24a, 24b, 24c downstream from the third chambers 23a, 23b, 23c contain a detection reagent, such as a CRISPR/Cas reagent. In the illustrated embodiment, the first channel fourth chamber 24a and the third channel fourth chamber 24c are illustrating a positive signal for a pathogen/molecule and the second channel fourth chamber 24b is illustrating a negative signal for the pathogen/molecule.

An alternative configuration to FIG. 11A would split the sample after lysis for three chambers, each containing amplification reagents, and then down to three subsequent chambers containing CRISPR/Cas reagents and detection reagents. This second configuration may simplify the RPA by reducing the potential for interference between multiple amplification primers.

It will be understood that, although the terms first, second, etc., may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the term “and/or” includes any and all combinations of one or more of the associated listed items.

It will be understood that when an element is referred to as being “on” another element, it can be directly on the other element or intervening elements may also be present. In contrast, when an element is referred to as being “directly on” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between”, “adjacent” versus “directly adjacent”, etc.).

It will be understood that a kit using the FAST technology may contain parallel fluid passageways as shown in FIG. 1E to include detection of a positive control.

Relative terms such as “below” or “above” or “upper” or “lower” or “horizontal” or “vertical” may be used herein to describe a relationship of one element, layer or region to another element, layer or region as illustrated in the figures. It will be understood that these terms are intended to encompass different orientations of the device in addition to the orientation depicted in the figures.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising,” “includes” and/or “including” when used herein, specify the presence of stated features, operations, elements, and/or components, but do not preclude the presence or addition of one or more other features, operations, elements, components, and/or groups thereof.

Aspects and elements of all of the embodiments disclosed above can be combined in any way and/or combination with aspects or elements of other embodiments to provide a plurality of additional embodiments.

That which is claimed is:

1. A test device, comprising:
a body;
at least one fluid passageway extending within the body, the at least one fluid passageway comprising an inlet and a plurality of chambers in adjacent, spaced-apart relationship, the at least one fluid passageway configured for sequential passage of a fluid sample through at least some of the plurality of chambers; and
a plurality of isolation valves, each isolation valve selectively operable to control fluid flow into and out of one or more of the plurality of chambers.

2. The device of claim 1, wherein each isolation valve comprises a port extending within the body transverse to the at least one fluid passageway and a rod slidably or rotatably movable within the port.

3. The device of claim 1, wherein at least one of the plurality of chambers is funnel- or flute-shaped to facilitate fluid flow into an adjacent, downstream chamber.

4. The device of claim 2, wherein each rod is a carbon fiber rod.

5. The device of claim 2, wherein the port has a polygonal cross-sectional configuration, and wherein the rod has a corresponding polygonal cross-sectional configuration such that the rod is snugly movable within the port.

6. The device of claim 2, wherein each rod has visual indicia to correlate to a respective chamber.

7. The device of claim 1, wherein the body comprises a translucent or transparent polymeric material.

8. The device of claim 7, wherein the polymeric material is polydimethylsiloxane (PDMS).

9. The device of claim 1, wherein the plurality of chambers comprise a first chamber configured to receive an input sample, a second chamber containing an amplification reagent, and a third chamber containing a detection reagent.

10. The device of claim 9, wherein the detection reagent includes a CRISPR/Cas reagent.

11. The device of claim 9, further comprising a fourth chamber containing a detection reagent configured to undergo a visually detectable change.

12. The device of claim 1, wherein the plurality of chambers comprise a chamber configured to receive an input sample, and a detection chamber containing a detection reagent configured to undergo a visually detectable change.

13. The device of claim 1, wherein the body comprises indicia that indicates a proper orientation of the device during use.

14. The device of claim 1, wherein the at least one fluid passageway comprises a plurality of fluid passageways in adjacent, spaced apart relationship.

15. The device of claim 1, wherein the at least one fluid passageway comprises two or more fluid passageways that are used to test for a presence of two or more target molecules.

16. The device of claim 1, wherein the body is configured to be opened such that the fluid sample can be accessed from one of the plurality of chambers.

17. The device of claim 16, wherein the body is configured to be opened such that a detection strip can be dipped in the fluid sample or such that a pipette can obtain the fluid sample.

18. A method of positively detecting a presence of a target molecule within a fluid sample via a handheld device, the device including a plurality of chambers in adjacent, spaced-apart relationship, and a plurality of isolation valves selectively operable to control fluid flow into and out of at least some of the plurality of chambers, the method comprising:
providing the fluid sample into a first chamber of the device;
opening a first one of the isolation valves to allow the fluid sample to flow from the first chamber into a second chamber, the second chamber containing an amplification reagent;
opening a second one of the isolation valves to allow the fluid sample to flow from the second chamber into a third chamber, the third chamber containing a detection reagent;
illuminating the fluid sample in the third chamber with light at a fluorescence excitation wavelength; and
determining whether the fluid sample in the third chamber fluoresces in response to the light.

19. The method of claim 18, wherein opening the first one of the isolation valves comprises laterally sliding or rotating a rod of the first one of the isolation valves, and wherein opening the second one of the isolation valves comprises laterally sliding or rotating a rod of the second one of the isolation valves.

20. The method of claim 18, further comprising heating the fluid sample within the second chamber prior to opening the second one of the isolation valves to allow the fluid sample to flow from the second chamber into the third chamber.

21. The method of claim 18, wherein the fluid sample includes a magnetic bead, and wherein the method further comprises opening a third one of the isolation valves to allow the fluid sample to flow from the third chamber to a fourth chamber, wherein a magnet is positioned within the fourth chamber or within a passageway between the third chamber and the fourth chamber.

22. The method of claim 18, wherein the detection reagent is a CRISPR/Cas reagent.

23. The method of claim 21, wherein the fourth chamber contains a reagent configured to undergo a colorimetric change.

24. A test kit for analyzing animal and/or human samples, the test kit comprising:
a test device; and
a device for collecting a sample;
the test device comprising:
a fluid passageway with a plurality of pre-filled chambers; and
a plurality of isolation valves, each isolation valve selectively operable to control sample flow between adjacent chambers.

25. The test kit of claim 24, further comprising a vial containing a substance configured to disrupt a pathogen/molecule in the sample.

26. The test kit of claim 24, further comprising a warming device configured to warm the sample.

27. The test kit of claim 26, wherein the warming device is configured to excite a reaction by externally providing activation energy.

28. The test kit of claim 26, wherein the warming device comprises a pouch of chemically activatable material.

29. The test kit of claim 24, further comprising a device configured to illuminate the sample with light at a fluorescence excitation wavelength.

30. A method of positively detecting a presence of a target molecule within a fluid sample, the method comprising:
providing the fluid sample into a first chamber of a handheld device, the device including a plurality of chambers in adjacent, spaced-apart relationship, and a plurality of isolation valves selectively operable to control fluid flow into and out of at least some of the plurality of chambers;
opening a first one of the isolation valves to allow the fluid sample to flow from the first chamber into a second chamber, the second chamber containing an amplification reagent or other reagent; and
opening a second one of the isolation valves to allow the fluid sample to flow onto a detection strip, or into an analytical device, or into a container for transfer to the analytical device.

31. The method of claim 30, wherein opening the first one of the isolation valves comprises laterally sliding or rotating a rod of the first one of the isolation valves, and wherein opening the second one of the isolation valves comprises laterally sliding or rotating a rod of the second one of the isolation valves.

32. The method of claim 30, further comprising heating the fluid sample within the second chamber prior to opening the second one of the isolation valves.
